(19) United States
(12) Patent Application Publication
(10) Pub. No.: US 2017/0042780 A1
(43) Pub. Date: Feb. 16, 2017

(54) DENTIFRICE COMPOSITION WITH GUM BLEEDING RELIEF EFFICACY

(71) Applicant: COLGATE-PALMOLIVE COMPANY, New York, NY (US)

(72) Inventors: Shaopeng Xu, Guangzhou (CN); Peng Yan, Guangzhou (CN)

(21) Appl. No.: 15/234,362

(22) Filed: Aug. 11, 2016

(30) Foreign Application Priority Data
Aug. 13, 2015 (CN) 201510587338.6

(51) Int. Cl. A61K 8/44; A61K 31/195; A61K 8/21; A61Q 11/00

(52) U.S. Cl. CPC A61K 8/445; A61Q 11/00; A61K 31/195; A61K 8/21; A61K 2800/30; A61K 2800/78; A61K 2800/28

(57) ABSTRACT

The present invention provides an oral care composition comprising 4-(aminomethyl)benzoic acid in an amount of from 0.05 weight % to 5 weight %, based on the total weight of the composition.

### CROSS-REFERENCE TO RELATED PATENT APPLICATIONS

This application claims the benefit of priority under 35 U.S.C. 119(a) to Chinese Patent Application Serial No. 201510587338.6 filed Aug. 13, 2015, the entirety of which is incorporated herein by reference.

### BACKGROUND

Bleeding gums are a common oral problem, and can be a sign of gingivitis and other oral conditions and may lead to serious medical problems if not addressed. Bleeding of the gums is caused by a build-up of plaque, which generates toxins that cause inflammation of the soft gum tissue. Many dentifrice products have been launched worldwide claiming gum bleeding relief efficacy, such as Yun Nan Bai Yao Toothpaste in China and Paradontox Toothpaste in Phillippines. Antibacterial agents have been used in oral care products to reduce plaque and gingivitis, and hence to relieve gum bleeding. Although the coagulant tranexamic acid has been used in oral care products to treat bleeding gums, its use in such compositions is limited due to potential thromboembolic risk and other drug interactions. It therefore would be desirable to provide an alternative oral care composition which can treat or inhibit bleeding of the gums.

### BRIEF SUMMARY

In a first aspect, the present invention provides an oral care composition comprising 4-(aminomethyl)benzoic acid in an amount of from 0.05 weight % to 5 weight %, based on the total weight of the composition.

Optionally, the 4-(aminomethyl)benzoic acid is present in an amount of from 0.1 weight % to 1 weight %, based on the total weight of the composition. Further optionally, the 4-(aminomethyl)benzoic acid is present in an amount of from 0.1 weight % to 0.5 weight %, based on the total weight of the composition. Still further optionally, the 4-(aminomethyl)benzoic acid is present in an amount of about 0.3 weight %, based on the total weight of the composition.

Optionally, the composition is free from tranexamic acid.

Optionally, the oral care composition further comprises a fluoride ion source, an abrasive, an antibacterial agent, an anticalculus agent, or an antisensitivity agent, or a combination of any two or more thereof.

Optionally, the oral care composition further comprises a diluent, a foam modulator, a thickening agent, a viscosity modifier, a humectant, a sweetener, a flavorant or a pigment, or a combination of any two or more thereof.

Optionally, the composition is a toothpaste, tooth gel, mouth wash, mouth rinse, spray, strip, chewing gum, lozenge, chewable tablet, dissolvable tablet, or confectionary item.

In a second aspect, the present invention provides an oral care composition of the present invention for use in a method of reducing or inhibiting gum bleeding.

In a third aspect, the present invention provides a method of reducing or inhibiting gum bleeding, the method comprising applying an effective amount of the oral care composition of the present invention to the oral cavity of a subject in need thereof.

In a fourth aspect, the present invention provides 4-(aminomethyl)benzoic acid for use in reducing or inhibiting gum bleeding.

In a fifth aspect, the present invention provides the use of 4-(aminomethyl)benzoic acid to reduce or inhibit gum bleeding.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material. Unless otherwise specified, all ratios expressed herein and elsewhere in the specification should be understood to refer to ratios by weight.

As discussed above, it would be desirable to provide oral care compositions which can treat or inhibit bleeding of the gums.

The present invention therefore provides, in a first aspect, an oral care composition comprising 4-(aminomethyl)benzoic acid in an amount of from 0.05 weight % to 5 weight %, based on the total weight of the composition. The present inventors have surprisingly found that 4-(aminomethyl)benzoic acid has a similar clotting time to tranexamic acid in the In-vitro Prothrombin Time (PT) Test (see Examples, below), but has been found to be safe for oral care applications.

In some embodiments, the oral care composition is free from tranexamic acid. By “free from tranexamic acid”, it is meant that the composition contains less than 0.05 weight % tranexamic acid, contains less than 0.01 weight % tranexamic acid, contains substantially no tranexamic acid, or contains no tranexamic acid (i.e. contains 0 weight % tranexamic acid).

In certain embodiments, the 4-(aminomethyl)benzoic acid is present in an amount of from 0.1 weight % to 1 weight %, from 0.1 weight % to 0.75 weight %, from 0.1 weight % to 0.5 weight %, from 0.2 weight % to 0.4 weight %, or about 0.3 weight %, based on the total weight of the composition.

In some embodiments, the oral care composition is a toothpaste, tooth gel, mouth wash, mouth rinse, spray, strip, chewing gum, lozenge, chewable tablet, dissolvable tablet, or confectionary item.

In various embodiments, the oral care composition further comprises a fluoride ion source, an abrasive, an antibacterial agent, an anticalculus agent, or an antisensitivity agent, or a combination of any two or more thereof. Examples of fluoride ion sources which may be used include sodium fluoride and sodium monofluorophosphate. Examples of abrasives which may be used include silica abrasives. Examples of antibacterial agents which could be used include triclosan, zinc oxide and zinc citrate. Examples of anticalculus agents which could be used include tetrapotassium pyrophosphate and tetrasodium pyrophosphate. An example of an antisensitivity agent which could be used is potassium nitrate.

The oral care composition may also further comprise a diluent, a foam modulator, a thickening agent, a viscosity modifier, a humectant, a surfactant, a sweetener, a flavorant or a pigment, or a combination of any two or more thereof. Examples of diluents which could be used include water and ethanol. Examples of foam modulators which could be used include polyethylene glycols. Examples of thickening agents which could be used include cellulose gums. Examples of viscosity modifiers which could be used include clays and silicas. Examples of humectants which could be used include polyhydric alcohols. Examples of surfactants which could be used include anionic surfactants, nonionic surfactants and cationic surfactants.

In a second aspect, the present invention provides an oral care composition according to any of the above-described embodiments for use in a method of reducing or inhibiting gum bleeding.

In a third aspect, the present invention provides a method of reducing or inhibiting gum bleeding, the method comprising applying an effective amount of an oral care composition according to any of the above-described embodiments to the oral cavity of a subject in need thereof.

In a fourth aspect, the present invention provides 4-(aminomethyl)benzoic acid for use in reducing or inhibiting gum bleeding. In certain embodiments, the 4-(aminomethyl)benzoic acid may be comprised in a composition in an amount of from 0.05 weight % to 5 weight %, from 0.1 weight % to 1 weight %, from 0.1 weight % to 0.75 weight %, from 0.1 weight % to 0.5 weight %, from 0.2 weight % to 0.4 weight %, or about 0.3 weight %, based on the total weight of the composition.

In a fifth aspect, the present invention provides the use of 4-(aminomethyl)benzoic acid to reduce or inhibit gum bleeding. In certain embodiments of such a use, the 4-(aminomethyl)benzoic acid may be comprised in a composition in an amount of from 0.05 weight % to 5 weight %, from 0.1 weight % to 1 weight %, from 0.1 weight % to 0.75 weight %, from 0.1 weight % to 0.5 weight %, from 0.2 weight % to 0.4 weight %, or about 0.3 weight %, based on the total weight of the composition.

### EXAMPLES

### Example 1

An In-vitro Prothrombin Time (PT) Test has been designed to test the coagulant efficacy (see Luz, L. et at., International Journal of Biological Macromolecules, 58 (2013), p. 31-36).

The coagulant efficacy of the compositions and ingredients of the present invention was tested against that of tranexamic acid and two controls: deionized (DI) water, and a solution of 0.9 weight % sodium chloride in water (hereinafter referred to as “saline solution”). The experimental methodology used was:

1) Sample Preparation

When testing the “Coagulant Ingredients” tranexamic acid and 4-(aminomethyl)benzoic acid in Table 1, below, the active ingredient was mixed with water at a 1:100 ratio by weight, to give a concentration of 1.0 weight % (to one decimal place) of the ingredient under test. When testing the “Toothpastes” in Table 2, below, a toothpaste slurry was prepared by mixing 1 part of the toothpaste under test with 2 parts DI water (all parts being parts by weight); then the supernatant was obtained by centrifuging the slurry for 20 mins at 8000 rpm.

2) Prothrombin Time (PT) Test

0.1 mL of experimental animal plasma was placed in each testing tube (animal blood samples used in the experiment were waste products, and the animals were otherwise used for food). 0.05 mL of the test samples (0.05 mL of DI water or saline solution in control group) were added to separate tubes and mixed with the plasma. The mixtures were then heated in a water bath at 37° C. for 3 min, then 0.2 mL of thromboplastin (which had been preheated to 37° C.) was added to each testing tube and the contents mixed. A stopwatch was started immediately upon adding the thromboplastin to record the coagulation time (Prothrombin Time). The experiment was repeated 10 times for each sample, and the mean Prothrombin Time calculated. The results are shown in Tables 1 and 2, below:

TABLE 1

| | Coagulant Ingredient | | | |
|---|---|---|---|---|
| | | | 4-(aminomethyl) | Tranexamic |
| | DI | Saline | benzoic | acid |
| Sample | water | solution | acid (1 weight %) | (1 weight %) |
| Prothrombin Time (s) | 19.37 | 17.40 | 11.14 | 11.66 |

TABLE 2

Toothpaste

| | | Toothpaste w. 0.3 weight % |
|---|---|---|
| | Control | 4-(aminomethyl) |
| Sample | toothpaste | benzoic acid |
| Prothrombin Time (s) | 22.69 | 10.57 |

As can be seen in Table 1, 4-(aminomethyl) benzoic acid has significantly lower clotting time than the controls (DI water and saline solution). No significant difference in clotting time was observed between tranexamic acid and 4-(aminomethyl) benzoic acid.

In the toothpaste sample study in Table 2, it was shown that the toothpaste containing 0.3 weight % 4-(aminomethyl)benzoic acid has a significantly lower Prothrombin time than the control toothpaste.

These results indicate that 4-(aminomethyl) benzoic acid can accelerate the in-vitro coagulation process, and can be applied in toothpaste to deliver gum bleeding relief efficacy.

### Example 2

An example of a toothpaste containing 4-(aminomethyl)benzoic acid is given in Table 3, below:

TABLE 3

| Ingredient | Weight % |
|---|---|
| 4-(aminomethyl)benzoic acid | 0.1-1 |
| Humectants | 30-50 |
| Thickening agents | 5-15 |
| Abrasives | 5-20 |
| Surfactants | 5-15 |
| Fluoride ion source | 0.1-1 |
| Anticalculus agents | 0.1-5 |
| Antibacterial agents and | 0.5-5 |
| preservatives | |
| Water and minors | 10-40 |

1. An oral care composition comprising 4-(aminomethyl)benzoic acid in an amount of from 0.05 weight % to 5 weight %, based on the total weight of the composition.

2. The oral care composition of claim 1, wherein the 4-(aminomethyl)benzoic acid is present in an amount of from 0.1 weight % to 1 weight %, based on the total weight of the composition.

3. The oral care composition of claim 2, wherein the 4-(aminomethyl)benzoic acid is present in an amount of from 0.1 weight % to 0.5 weight %, based on the total weight of the composition.

4. The oral care composition of claim 3, wherein the 4-(aminomethyl)benzoic acid is present in an amount of about 0.3 weight %, based on the total weight of the composition.

5. The oral care composition of claim 1, wherein the composition is free from tranexamic acid.

6. The oral care composition of claim 1, further comprising a fluoride ion source, an abrasive, an antibacterial agent, an anticalculus agent, or an antisensitivity agent, or a combination of any two or more thereof.

7. The oral care composition of claim 1, further comprising a diluent, a foam modulator, a thickening agent, a viscosity modifier, a humectant, a sweetener, a flavorant or a pigment, or a combination of any two or more thereof.

8. The oral care composition of claim 1, wherein the composition is a toothpaste, tooth gel, mouth wash, mouth rinse, spray, strip, chewing gum, lozenge, chewable tablet, dissolvable tablet, or confectionary item.

9. The oral care composition of claim 1 for use in a method of reducing or inhibiting gum bleeding.

10. A method of reducing or inhibiting gum bleeding, the method comprising applying an effective amount of the oral care composition of claim 1 to the oral cavity of a subject in need thereof.

11-12. (canceled)
